(19) United States
(12) Patent Application Publication
(10) Pub. No.: US 2025/0236590 A1
(43) Pub. Date: Jul. 24, 2025

(54) A METHOD FOR THE PREPARATION OF INDOLE-3-CARBOXYLIC ACID DERIVATIVES

(71) Applicant: Indubhusan DEB

(72) Inventor: Indubhusan DEB

(21) Appl. No.: 18/702,220

(22) PCT Filed: Oct. 20, 2022
(86) PCT No.: PCT/IN2022/050940

§ 371 Date: Apr. 17, 2024

(51) Int. Cl. C07D 209/42; C07B 59/00; C07D 401/12; C07D 417/12

(52) U.S. Cl. CPC C07D 209/42; C07B 59/002; C07D 401/12; C07D 417/12

(57) ABSTRACT

The present invention provides a new synthetic method for the preparation of building block indole-3-carboxylic acid (ICA) derivatives used as a key starting material to produce several artificial drugs. An efficient, safe, operationally simple, cost-effective method for the preparation of several ICA derivatives has been introduced in a very simple way. Utilizing easily available starting material, reagent, and solvent several key starting materials of ICA derivatives have been synthesized rapidly indicates the novel method is functional group tolerable. The preparation process does not involve use of any reducing agents or transition metals in the economically viable and operationally simple condition in one pot. ICA derivatives have been generated from isatin derivative in one step and this methodology is important with a wide range of applications from the drug development and material synthesis CN point of view.

### FIELD OF THE INVENTION

The present invention relates to a synthetic method for the preparation of building block indole-3-carboxylic acid (ICA) derivatives used as a key starting material to produce several artificial drugs.

### BACKGROUND OF THE INVENTION

Indole-3-carboxylic acid (ICA) derivatives are building block motif as it is widely present in numerous natural products, and has been used to produce several artificial drugs such as Tropisetron, Dolasetron, etc. or it's derivatives are broadly used as (a) anticancer agents (CPI-1205), (b) serotonin 5-HT4 and 5-HT6 antagonists, (c) EphB3 receptor tyrosine kinase inhibitors, and (d) potential therapeutic agents for Alzheimer's disease. There are several problems in the existing synthetic method for the preparation of ICA derivatives such as the requirement of expensive transition metal catalyst, designed directing group preparation, and multistep operation. Indeed, there is no direct route for the synthesis of this key intermediate by which it could be produced smoothly in a short time from an easily available starting material that could be solved by the present invention.

References may be made to Journal “Org. Lett. 2018, 20, 4540-4544” wherein a sequential Corey-Chaykovsky reactions of isatins, spiroepoxy-, or spiroaziridine oxindoles with sulfur ylide have led to the discovery of a unique reaction mode that allows easy and direct one-pot access to a range of spiro cyclopropyl oxindoles.

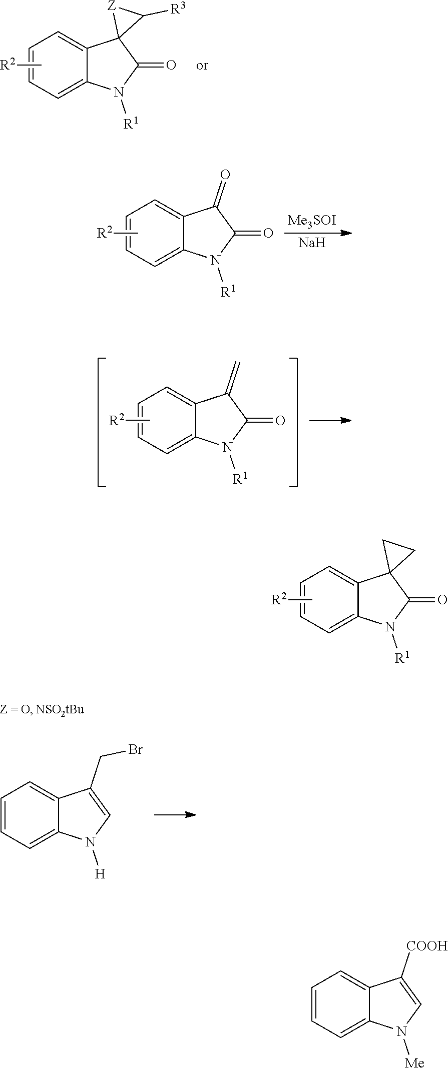

References may be made to Journal “Chem Heterocycl Compd 19, 40-42 (1983)”, wherein a simple method for the preparation of 3-indolylglyoxals, two new methods for the synthesis of indolylglyoxal structures were proposed. The first method consists of 3-hydroxy-acetyl indole oxidation with the dimethyl sulfoxide-oxalyl chloride complex, but for some reason, it is not properly applicable to the preparation of substituted indolylglyoxals. In the second case, the readily synthesized 3-indolylglyoxyl chlorides are reduced to the corresponding aldehydes in good yields employing trialkyltin hydrides.

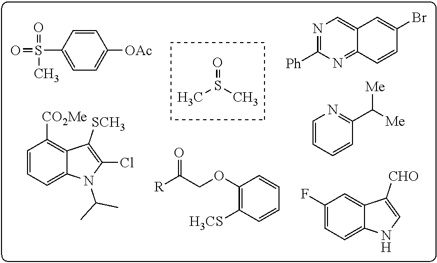

References may be made to Journal “Synthesis 2016; 48(10): 1421-1436”, wherein Dimethyl sulfoxide is generally characterized as a solvent and oxidant rather than as a substrate, building block, or synthon in organic chemistry. However, an abundance of reports has recently appeared that demonstrate dimethyl sulfoxide acting in these roles. This review article offers a comprehensive summary of the literature on this topic until the end of 2015. Synthetic transformations that have utilized the ‘C—S—C’, ‘C’, and ‘C—S’ fragments of dimethyl sulfoxide as building blocks are systematically summarized. Recent Highlights of DMSO-Based Oxidations, DMSO-Based Methylthiomethylation (—CH₂SMe), DMSO as a One-Carbon Synthon, DMSO-Based Methylation (+Me), DMSO-Based Methylenation (—CH₂—), DMSO-Based Annulation/Aromatization (═CH—), DMSO-Based Formylation (—CHO), DMSO-Based Cyanation (—CN), DMSO as Synthon for ‘S—C’ Functionalities, DMSO-Based Thiomethylation (—SMe), DMSO-Based Methylsulfonylation (—SO₂Me).

References may be made to Journal “Adv. Synth. Catal. 2020, 362, 65-86”, wherein Dimethyl sulfoxide (DMSO) has a long history of use as a polar solvent and active pharmaceutical ingredient in the past decades. However, in this decade DMSO has attracted attention. Dimethyl sulfoxide (DMSO) has a long history of use as a polar solvent and active pharmaceutical ingredient in the past decades. However, in this decade DMSO has attracted the attention of scientists as a source of oxygen, carbon, or sulfur in a wide range of organic synthesis. In this review, the latest findings in this area based on the application of DMSO as a single- or a dual-synthon were classified and summarized.

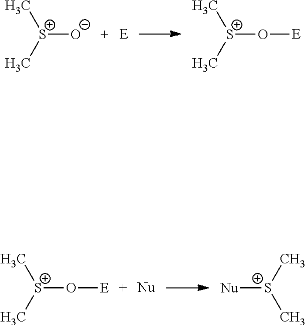

References may be made to Journal “Synthesis 1981; 1981(3): 165-185”, wherein selected examples of the use of activated dimethyl sulfoxide reagents in organic synthesis are discussed with the emphasis being placed on low-temperature studies. Reactions of acetic anhydride, oxalyl chloride, t-butyl hypochlorite, or halogens (among others) with dimethyl sulfoxide at appropriate temperatures yield intermediate dimethyl sulfonium salts. These salts were particularly useful in the synthesis of sulfinimines and sulfoximines and the selective oxidation of structurally diverse alcohols to the corresponding carbonyl compounds.

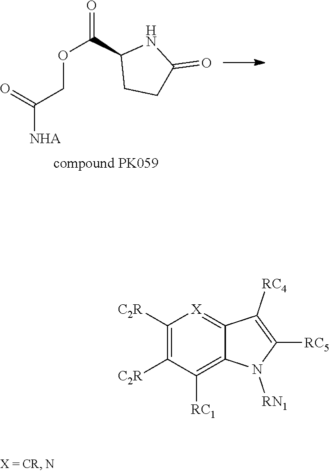

References may be made to patent application “US 20110059953 A1, wherein synthesis of several biologically active compounds employing N-(9-Ethyl-9H-carbazol-3-yl)-2,2,2-trifluoro-acetamide or indole derivative 1H-indole-3-carboxamide for treating a subject who has a lesion or a tumour in which p53 carries a Y220C mutation was reported. Their invention relates to compounds that can bind to p53 protein molecules.

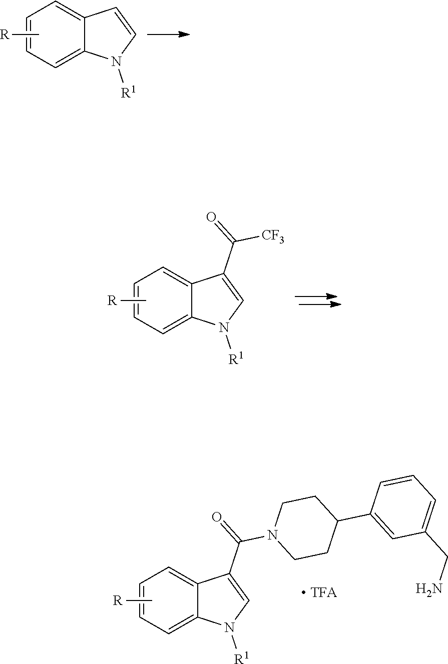

References may be made to Journal “Bioorg. Med. Chem. Lett. 15 (2005) 2734-2737”, wherein a new series of novel mast cell tryptase inhibitors is reported, which features the use of an indole structure as the hydrophobic substituent on an m-benzylaminepiperidine template. The best members of this series display well in vitro activity and excellent selectivity against other serine proteases. They have reported the synthesis and SAR evaluation of a novel class of small-molecule mast cell tryptase inhibitors. The compounds are an extension of their tryptase program and are very potent, orally bioavailable inhibitors.

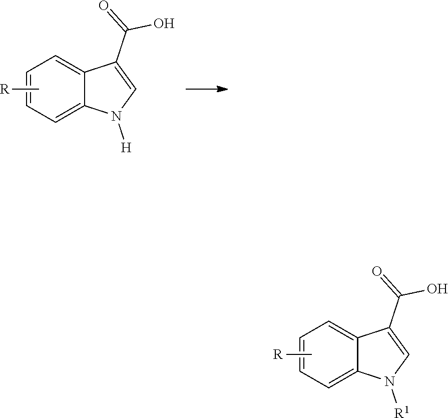

References may be made to Patent application JP 2001261642 A, showed the use of several indole-3-carboxylic acid derivatives as a raw material for medicines and agricultural chemicals purpose in their recent report.

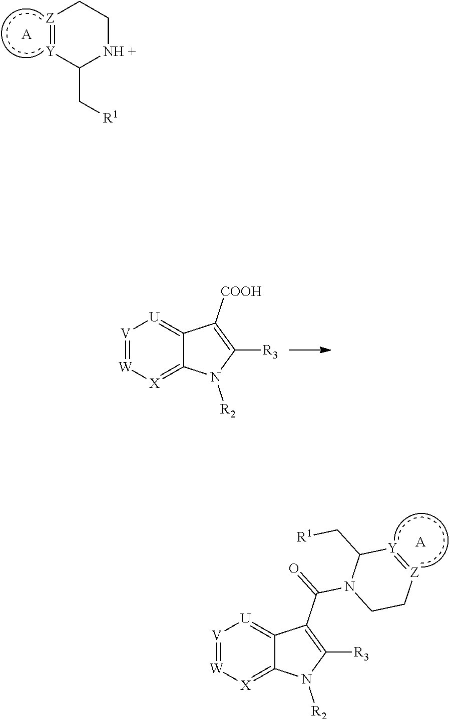

References may be made to Patent application WO 2012/114252 A1, in which many novel indole and pyrrolopyridine amide derivatives and their use as pharmaceuticals has been reported. The invention also concerns related aspects including processes for the preparation of the compounds, pharmaceutical compositions containing one or more compounds, and especially their use as orexin receptor antagonists. Their compounds have been utilized for the prevention or treatment of diseases like sleep disorders, stress-related syndromes, addictions, cognitive and psychiatric and neurologic disorders, eating or drinking disorders.

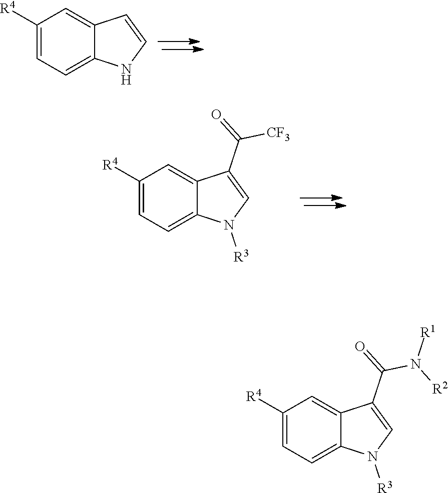

References may be made to Patent application WO 2014/172759 A1, wherein a remarkable work producing different important amide derivatives employing substituted indole-3-carboxylic acid as intermediate starting from indole derivative. These amide compounds are useful in the positive modulation of the alpha 7 nicotinic acetylcholine receptor (α₇ nAChR). The invention also relates to the use of these compounds in the treatment or prevention of a broad range of diseases in which the positive modulation of al nAChR is advantageous, including neurodegenerative neuropsychiatric diseases and also inflammatory diseases.

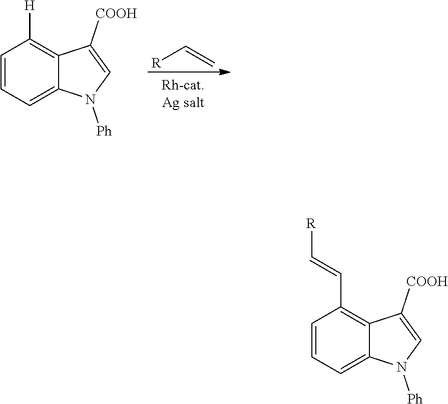

References may be made to Journal “J. Org. Chem. 2018, 83, 5639-5649”, wherein Okada et al. have demonstrated that N-phenylindole-3-carboxylic acids undergo alkenylation at the C-4 position on treatment with alkenes such as acrylate ester, acrylamide, and acrylonitrile in the presence of a rhodium(III) catalyst and a silver salt oxidant via regioselective C—H bond cleavage. The information obtained in this work would help design new catalytic substitution reactions on benzo-fused heteroarenes of important medicinal and materials chemistry.

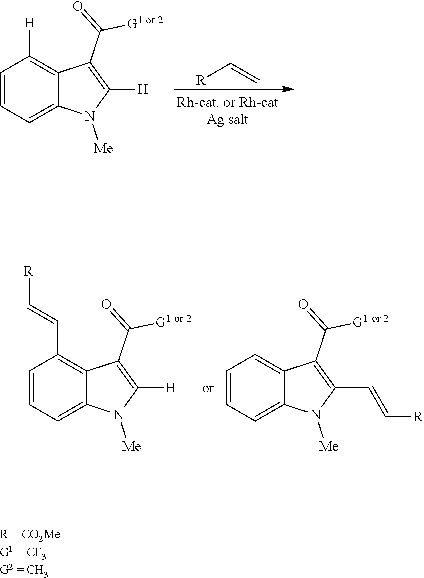

References may be made to Journal “Org. Lett. 2016, 18, 5496-5499”, wherein a novel mode of achieving site selectivity between C-2 and C-4 positions in the indole framework by altering the property of the ketone directing group has been disclosed. Methyl ketone, as the directing group, furnishes exclusively C-2 alkenylated product, whereas trifluoromethyl ketone changes the selectivity to C-4, indicating that the electronic nature of the directing group controls the unusual choice between a 5-membered and a 6-membered metallacycle. The screening of other carbonyl-derived directing groups reveals that strong and weak directing groups exhibit opposite selectivity.

Several other methods are reported in the academic literature regarding the synthesis of indole-3-carboxylic acid derivatives. The synthetic routes are described into four different categories mainly: a) C—H bond activation strategy, b) C—Br bond activation strategy, c) electrophilic substitution strategies, and d) electron catalyzed C—N bond formation strategy.

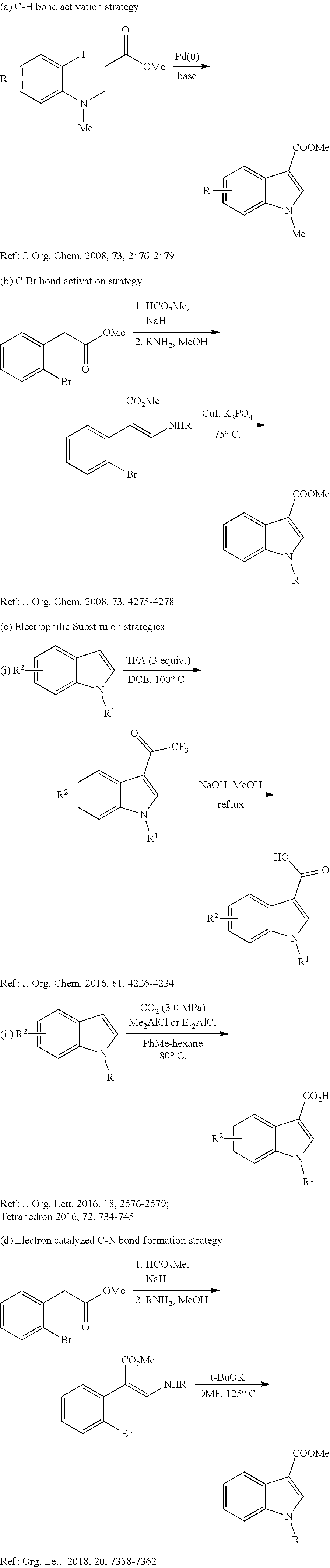

In recent times C—H bond activation strategy is an important tool for various synthetic transformations but it has some limitations such as the essentiality of transition metal and in most cases, it is not applicable for large-scale preparation.

References may be made to Journal “J. Org. Chem. 2008, 73, 2476-2479”, in which β-(2-iodoanilino) esters derivatives could be converted to indole-3-carboxylic acid ester derivatives via intramolecular α-arylation in presence of a catalytic amount of Pd(PPh₃)₄ and potassium phenoxide. Preparation of the directing group from 2-iodoaniline derivative and methyl acrylate is another demerit, and the use of Pd on an industrial scale is not economical also. Moreover, the yield was poor in this intramolecular α-arylation of β-(2-iodoanilino)esters reaction.

Applying Ullmann reaction, C—N bond formation methodology through C—X(X═Br) bond activation employing Cu catalyst is a good achievement for indole-3-carboxylic acid preparation. A variety of N-alkylated and N-arylated derivatives of methyl 1H-indole-3-carboxylate has been synthesized taking methyl 2-(2-bromophenyl)-2-formyl acetate with different primary amines employing Cu catalyst. [J. Org. Chem. 2008, 73, 4275-4278] Indeed, starting material methyl 2-(2-bromophenyl)acetate preparation is a multistep process and it is highly expensive, these are the major problem for industrial preparation. Many reagents and solvents such as methyl formate, primary amine, K₃PO₄, NaH, MeOH, and DMF were essential for this reaction. They didn't explore simple N—H free indole-3-carboxylate.

Another promising route to prepare indole-3-carboxylic acid derivative for large-scale purposes is the Friedel-Craft reaction of indole derivative. References may be made to Journal “J. Org. Chem. 2016, 81, 4226-4234”, wherein a Friedel-Craft reaction specifically trifluoroacetylation reaction of indole derivatives using trifluoroacetic acid at 100° C. to prepare indolyl trifluoromethyl ketone derivatives which is the precursor of indole-3-carboxylic acid has been reported. After hydrolysis of these ketone derivatives (reflux in MeOH in presence of NaOH), they got indole-3-carboxylic acid derivative.

References may be made to Journal “Tetrahedron 72, (2016), 734-745” wherein another way of performing a Friedel-Craft reaction in presence of Lewis acid (Me₂AlCl or EtAlCl₂) under 3.0 MPa pressure of CO₂ was reported. They obtained a very poor yield of indole-3-carboxylic acid in cases of N—H free indole. The major drawbacks of these reactions for large scale industrial preparation are the requisite of various indole derivatives, expensive Lewis acid, and high-pressure reactions.

Electron catalyzed C—N bond formation methodology is a good development in synthetic chemistry nowadays. Recently, Karchava's group disclosed a new synthetic strategy based on electron catalyzed intramolecular C—N bond formation reaction for the synthesis of N-functionalized indole-3-carboxylates, using the t-BuOK/DMF system at 125° C. employing 3-amino-2-(2-bromphenyl)acrylate. [Org. Lett. 2018, 20, 7358-7362] Due to the shortcomings of complexity and the high cost of starting material and high-temperature reactions it is very difficult for industrial preparation.

All the previous literature reports show, the key intermediate indole-3-carboxylic acid derivatives have been prepared in an expensive multistep process. Therefore there is a dire need in the state of art for a simple yet efficient process for the synthesis of indole-3-carboxylic acid derivatives.

### OBJECTIVES OF THE INVENTION

The main object of the present invention is to provide a straightforward cost-effective one-pot synthesis of indole-3-carboxylic acid (ICA) derivatives in high yield utilizing cheaper starting material.

Another object of the present invention is to provide a process for the synthesis of indole-3-carboxylic acid (ICA) derivatives in which the overall reduction process (formation of ICA derivatives) carried out without treating any reducing agent externally in a mild condition.

Yet another object of the present invention is to provide a synthetic methodology for the synthesis of ICA derivatives and it's an application for the synthesis of commercially available drug Tropisetron.

Yet another object of the present invention is to provide a synthetic methodology for the synthesis of ICA derivatives under mild reaction conditions and in a very short reaction time (approx. 2 h).

Yet another object of the present invention is to provide a commercially viable process for the synthesis of ICA derivatives. The reaction proceeded smoothly in atmospheric pressure at the inert condition. No transition metal catalyst is required for this single-step conversion.

Still another object of the present invention is to provide operationally simple and well tolerable of various functional groups. Accordingly, the methodology is important for the production of several key intermediates of indole-3-carboxylic acid derivatives for the synthesis of commercially available drugs.

### BRIEF DESCRIPTION OF THE DRAWING

FIG. 1 represents direct synthesis of indole-3-carboxylic acid derivatives from isatin derivatives, wherein R₁ and R₂ are independently selected from the group consisting of hydrogen, linear or branched chain (C₁-C₁₂), perfluoro(C₁-C₁₂)alkyl, (C₃-C₁₂)cycloalkyl, (C₆-C₁₂)bicycloalkyl, (C₃-C₁₄)tricycloalkyl, (C₆-C₁₀)aryl, (C₆-C₁₀)aryl(C₁-C₆)alkyl, (C₁-C₆)alkyl(C₆-C₁₀)aryl, (C₆-C₁₀)aryl(C₁-C₃)alkoxy, perfluoro(C₆-C₁₀)aryl, perfluoro(C₆-C₁₀)aryl(C₁-C₃)alkyl, (C₅-C₁₀)heteroaryl, (C₅-C₁₀)heteroaryl(C₁-C₃)alkyl, hydroxy, (C₁-C₁₂)alkoxy, (C₃-C₁₂)cycloalkoxy, (C₆-C₁₂)bicycloalkoxy, (C7-C14)tricycloalkoxy, (C₆-C₁₀)aryloxy(C₁-C₃)alkyl, (C₆-C₁₀)aryloxy, (C5-C10)heteroaryloxy, (C₁-C₆)acyloxy, halogen, nitro and amino;

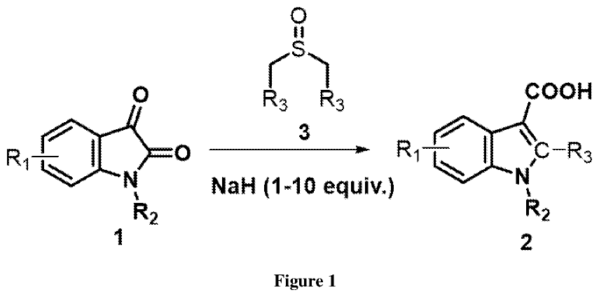

R₃ is selected from the group consisting of hydrogen, deuterium, and linear or branched chain (C₁-C₁₂);
C is carbon or ¹³ labelled carbon.

### SUMMARY OF THE INVENTION

Accordingly, the present invention provides a process for the synthesis of indole-3-carboxylic acid (ICA) compound of Formula 2

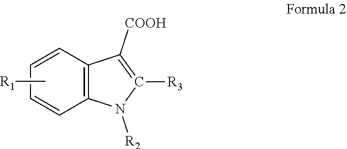

wherein
R₁ and R₂ are independently selected from the group consisting of hydrogen, linear or branched chain (C₁-C₁₂), perfluoro(C₁-C₁₂)alkyl, (C₃-C₁₂)cycloalkyl, (C₆-C₁₂) bicycloalkyl, (C₃-C₁₄)tricycloalkyl, (C₆-C₁₀)aryl, (C₆-C₁₀)aryl(C₁-C₆)alkyl, (C₁-C₆)alkyl(C₆-C₁₀)aryl, (C₆-C₁₀)aryl(C₁-C₃)alkoxy, perfluoro(C₆-C₁₀)aryl, perfluoro(C6-C10)aryl(C₁-C₃)alkyl, (C₅-C₁₀)heteroaryl, (C₅-C₁₀)heteroaryl(C₁-C₃)alkyl, hydroxy, (C1-C12)alkoxy, (C₃-C₁₂)cycloalkoxy, (C₆-C₁₂)bicycloalkoxy, (C₇-C₁₄)tricycloalkoxy, (C6-C10)aryloxy(C₁-C₃)alkyl, (C₆-C₁₀)aryloxy, (C₅-C₁₀)heteroaryloxy, (C₁-C₆)acyloxy, halogen, nitro and amino;
R₃ is selected from the group consisting of hydrogen, deuterium, and linear or branched chain (C₁-C₁₂);
C is carbon or ¹³ labeled carbon,
comprising the steps of:
degassing Isatin derivatives of Formula 1 and solvent of Formula 3 into a two-neck round bottom flask equipped with an electromagnetic stirrer by the Freeze-Pump-Thaw method to obtain degassed mixture;

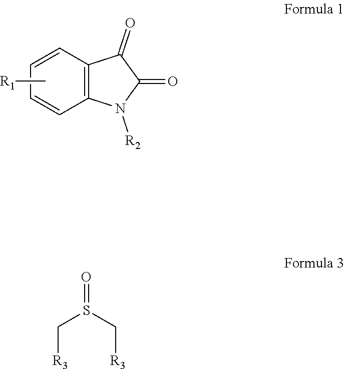

wherein R₁, R₂ and R₃ are same as above;
adding sodium hydride [NaH] in the degassed mixture as obtained in step (i) at room temperature in the range of 25 to 35° C. for a period in the range of 5 to 10 minutes followed by warming the mixture slowly to 70 to 100° C. at one atmospheric pressure kept for period in the range of 1 to 24 hours to obtain a solution;
cooling the solution as obtained in step (ii) at room temperature in the range of 25 to 35° C. followed by quenching with cold 1 N HCl solution;
extracting the organic part by EtOAc with brine, dried over Na₂SO₄, and concentrated under reduced pressure, purified by column chromatography on silica gel using petroleum ether/ethyl acetate (4:1) as eluent to obtain the compound of Formula I.

In an embodiment of the present invention, yield of compound of Formula 2 is in the range of 50-83%.

In another embodiment, the present invention provides a compound of Formula A

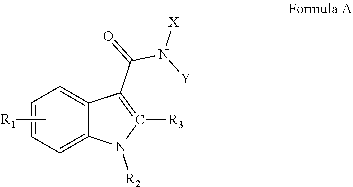

wherein
X═H or cyclohexyl;
Y═

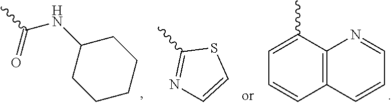

or
R₁ and R₂ are independently selected from the group consisting of hydrogen, linear or branched chain (C₁-C₁₂), perfluoro(C₁-C₁₂)alkyl, (C₃-C₁₂)cycloalkyl, (C₆-C₁₂) bicycloalkyl, (C₃-C₁₄)tricycloalkyl, (C₆-C₁₀)aryl, (C₆-C₁₀)aryl(C₁-C₆)alkyl, (C₁-C₆)alkyl(C₆-C₁₀)aryl, (C₆-C₁₀)aryl(C₁-C₃)alkoxy, perfluoro(C₆-C₁₀)aryl, perfluoro(C6-C10)aryl(C₁-C₃)alkyl, (C₅-C₁₀)heteroaryl, (C₅-C₁₀)heteroaryl(C₁-C₃)alkyl, hydroxy, (C1-C12)alkoxy, (C₃-C₁₂)cycloalkoxy, (C₆-C₁₂)bicycloalkoxy, (C₇-C₁₄)tricycloalkoxy, (C6-C10)aryloxy(C₁-C₃)alkyl, (C₆-C₁₀)aryloxy, (C₅-C₁₀)heteroaryloxy, (C₁-C₆)acyloxy, halogen, nitro and amino;
R₃ is selected from the group consisting of hydrogen, deuterium, and linear or branched chain (C₁-C₁₂);
C is carbon or ¹³ labeled carbon.

In yet another embodiment of the present invention, the compound of Formula A is selected from the group consisting of:

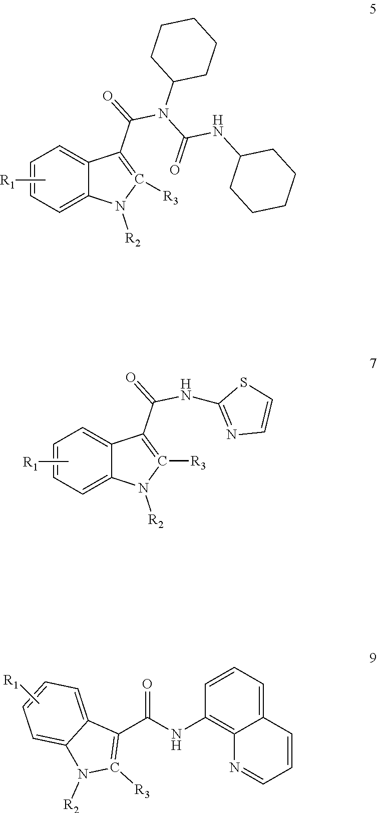

In yet another embodiment of the present invention, the compound of Formula 2 have been used to prepare commercially available drug Tropisetron (4) and potential bioactive indole compound of Formula A as disclosed herein.

In yet another embodiment of the present invention, said process is used to prepare C13-labelled ICA compound of Formula 2aa.

In yet another embodiment of the present invention, said process is used to prepare deuterated-ICA compound of Formula 2ab-2af.

In yet another embodiment of the present invention, in a process for the synthesis of ICA derivatives, various functional groups were well tolerated to explore in substrate scope, hence methodology is important for the production of several key intermediate indole-3-carboxylic acid derivatives.

In yet another embodiment of the present invention, NaH is used as a reagent under mild reaction conditions.

### DETAILED DESCRIPTION OF THE INVENTION

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below. The invention will now be described in detail in connection with certain preferred and optional embodiments, so that various aspects thereof may be more fully understood and appreciated.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

The present invention provides a synthetic method for the preparation of a key starting material indole-3-carboxylic acid (ICA) derivative. Indole-3-carboxylic acid (ICA) derivatives are building block motif as it is widely present in numerous natural products, has been used to produce several artificial drugs such as Tropisetron, Dolasetron, etc. or it's derivative broadly used as (a) anticancer agents (CPI-1205), (b) serotonin 5-HT4 and 5-HT6 antagonists, (c) EphB3 receptor tyrosine kinase inhibitors, (d) potential therapeutic agents for Alzheimer's disease.

The present invention provides one-pot production of indole-3-carboxylic acid derivative from isatin derivative and the process is straightforward. Sodium hydride has been used in mild conditions for this conversion, which has a lower price per kilogram compared to other reducing agents. Moreover, Isatin is a cheap starting material than indole/other functional directing groups used in the reported literature. The present invention discloses a process for obtaining indole-3-carboxylic acid derivative without use of any reducing agents or transition metals and hence is an one pot economically viable and operationally simple fast process suitable for industrial production. Accordingly the present invention provides a new methodology for the preparation of indole-3-carboxylic acid derivatives

An efficient, safe, operationally simple, cost-effective method for the preparation of several ICA derivatives has been introduced in a very simple way. Utilizing easily available starting material, reagent, and solvent several key starting materials of ICA derivatives have been synthesized rapidly indicates the novel method is well functional group tolerable. The unique feature of this straightforward methodology is the production of various novel ICA derivatives (overall reduced product) apart from treatment with any reducing agent or transition metal, in the economically viable and operationally simple condition in one pot. And this is the first report of whether ICA derivatives have been generated from isatin derivative in one step. Therefore, the research of this methodology is important with a wide range of applications from the drug development and material synthesis point of view.

There are several problems in the existing synthetic method for the preparation of ICA derivatives such as the requirement of expensive transition metal catalyst, designed directing group preparation, and multistep operation. Indeed, there is no direct route for the synthesis of this key intermediate by which it could be produced smoothly in a short time from an easily available starting material that could be solved by the present invention. In the present invention, only one step is involved to prepare the product from isatin derivative.

The present invention has been extended to synthesize a commercially available drug Tropisetron using indole-3-carboxylic acid as a key starting material.

The reaction was performed using N-protected isatin or its derivatives to prepare the building block key intermediate indole-3-carboxylic acid (ICA) derivatives.

In the present invention, several ICA derivatives could be prepared by utilizing the following procedure, where DMSO is used as a solvent as well as a reactant and NaH used as a base. Before the addition of NaH, the reaction mixture was subjected to degassing by the Freeze-Pump-Thaw method. After degassing, NaH was added portion-wise through a solid-additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution. The organic part was extracted by EtOAc with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl-1H-indole-3-carboxylic acid (2a) in 82% yield. To avoid column purification, the recrystallization method was carried out by dissolving crude reaction mixture in hot ethanol and the same yield was obtained.

In the present invention, no reducing agent, Lewis acid or transition metal catalysts have been used and various ICA derivatives could be formed in milder conditions (NaH in DMSO at 80° C.).

The reaction is fast (approx. 2 h) and straightforward, various functional group tolerated and could be performed smoothly at the optimal atmospheric conditions.

This one-pot operationally simple process is economically viable as it performs well in bottle-grade DMSO (should be free from dissolved oxygen) and large-scale operation.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### General Procedure for the Preparation of 1-Ethyl-1H-Indole-3-Carboxylic Acid (2a)

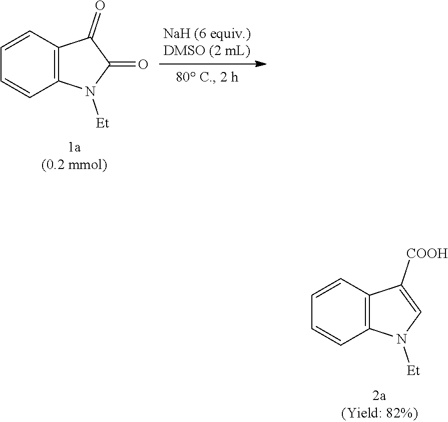 [SMILES: CCN1C(=O)C(=O)c2ccccc21 ; CCn1cc(C(=O)O)c2ccccc21]

### Milligram Scale Reaction

Into a 10 mL two neck round bottom flask equipped with an electromagnetic stirrer, transferred N-ethyl isatin (1 equiv., 0.2 mmol, 35 mg) and DMSO solvent (2 mL, 0.1 M) in an inert atmosphere. Then the whole reaction mixture was subjected to degassing by the Freeze-Pump-Thaw method. After degassing, NaH (Sodium hydride) (6 equiv., 1.2 mmol) was added portion-wise through a solid-additional funnel and stirred at room temperature (25 to 35° C.) for 10 minutes. After that, the reaction mixture was warmed slowly to 80° C. and was kept for 2 hours. After the full conversion was monitored by TLC, it was cooled to room temperature and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl-1H-indole-3-carboxylic acid (2a) in 82% yield (31 mg). To avoid column purification, recrystallization technique was carried out by dissolving crude reaction mixture in hot ethanol and the same yield was obtained.

### Gram Scale Reaction

Into a 100 mL two neck round bottom flask equipped with an electromagnetic stirrer, transferred N-ethyl isatin (1 equiv., 5.7 mmol, 1 g), and DMSO solvent (57 mL, 0.1 M) in an inert atmosphere. Then the whole reaction mixture was subjected to degassing by the Freeze-Pump-Thaw method. After degassing, NaH (6 equiv., 34.2 mmol) was added portion-wise through a solid-additional funnel and stirred at room temperature (30° C.) for 10 minutes. After that, the reaction mixture was warmed slowly to 80° C. and was kept for 2 hours. After the full conversion was monitored by TLC, it was cooled to room temperature and quenched with cold 1N HCl solution (200 mL). The organic part was extracted by EtOAc (3×100 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl-1H-indole-3-carboxylic acid (2a) in 75% yield (808 mg). To avoid column purification, the recrystallization method was carried out by dissolving crude reaction mixture in hot ethanol and the same yield was obtained.

The above-described procedure have been followed for the synthesis of 2b-2af. In cases of 1za, 1zb, and 1zc, deprotection happened during the reaction and obtained indole-3-carboxylic acid 2z.

| Starting Material (1a-1af) | | Desired Product with Yield (2a-2af) | |
|---|---|---|---|
| 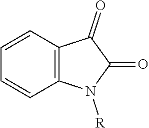 | 1a, R = Et 1b, R = Me 1c, R = Ph 1d, R = iPr 1e, R = Bn | 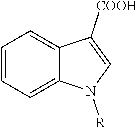 | 2a, R = Et, 82% 2b, R = Me, 52% 2c, R = Ph, 65% 2d, R = iPr, 45% 2e, R = Bn, 45% |
| 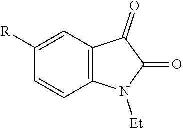 | 1f, R = Me 1g, R = F 1h, R = OCF₃ 1i, R = Cl | 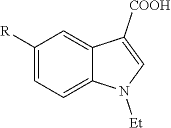 | 2f, R = Me, 83% 2g, R = F, 66% 2h, R = OCF₃, 55% 2i, R = Cl, 53% |
| 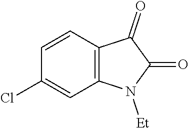 [SMILES: CCN1C(=O)C(=O)c2ccc(Cl)cc21] | 1j | 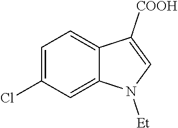 [SMILES: CCn1cc(C(=O)O)c2ccc(Cl)cc21] | 2j, 55% |
| 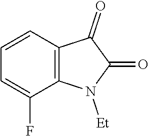 [SMILES: CCN1C(=O)C(=O)c2cccc(F)c21] | 1k | 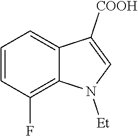 [SMILES: CCn1cc(C(=O)O)c2cccc(F)c21] | 2k, 58% |
| 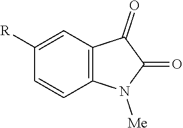 | 1l, R = Me 1m, R = F 1n, R = Cl 1o, R = Br | 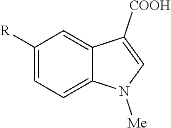 | 2l, R = Me, 58% 2m, R = F, 64% 2n, R = CL, 60% 2o, R = Br, 38% |
| 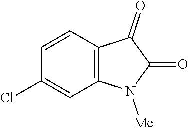 [SMILES: CN1C(=O)C(=O)c2ccc(Cl)cc21] | 1p | 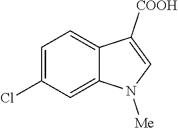 [SMILES: Cn1cc(C(=O)O)c2ccc(Cl)cc21] | 2p, 51% |
| 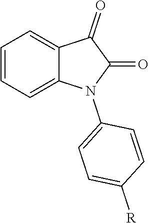 | 1q, R = Me 1r, R = ^{t}Bu 1s, R = OMe | 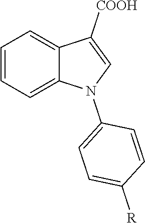 | 2q, R = Me, 76% 2r, R = ^{t}Bu, 76% 2s, R = OMe, 50% |
| 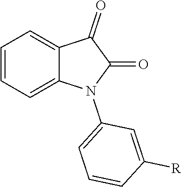 | 1t, R = Me 1u, R = F 1v, R = Cl 1w, R = OMe | 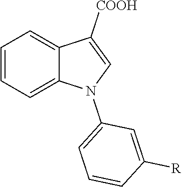 | 2t, R = Me, 80% 2u, R = F, 77% 2v, R = Cl, 37% 2w, R = OMe, 66% |
| 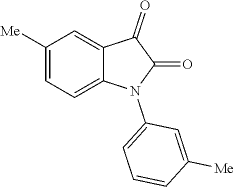 [SMILES: Cc1cccc(N2C(=O)C(=O)c3cc(C)ccc32)c1] | 1x | 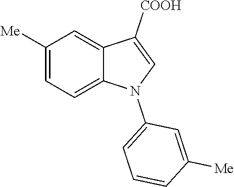 [SMILES: Cc1cccc(-n2cc(C(=O)O)c3cc(C)ccc32)c1] | 2x, 66% |
| 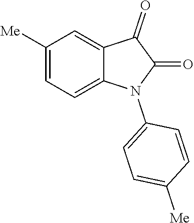 [SMILES: Cc1ccc(N2C(=O)C(=O)c3cc(C)ccc32)cc1] | 1y | 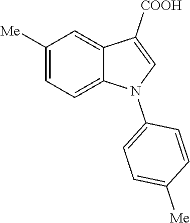 [SMILES: Cc1ccc(-n2cc(C(=O)O)c3cc(C)ccc32)cc1] | 2y, 80% |
| 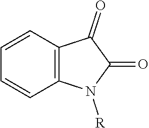 | 1za, R = —Boc 1zb, R = —Bn 1zc, R = allyl | 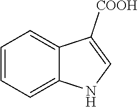 [SMILES: O=C(O)c1c[nH]c2ccccc12] | 2za, 47% 2zb, 40% 2zc, 46% 2z = 2za, 2zb, 2zc |
| 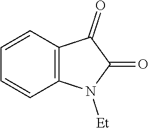 [SMILES: CCN1C(=O)C(=O)c2ccccc21] | 1aa | 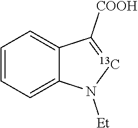 [SMILES: CCn1[13cH]c(C(=O)O)c2ccccc21] | 2aa, 70% |
| 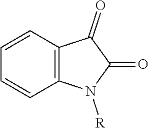 | 1ab, R = Et 1ac, R = Me 1ad, R = Ph | 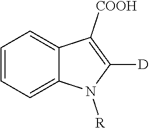 | 2ab, R = Et, 80% 2ac, R = Me, 62% 2ad, R = Ph, 54% |
| 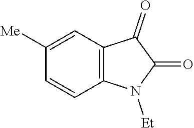 [SMILES: CCN1C(=O)C(=O)c2cc(C)ccc21] | 1ae | 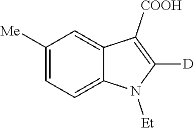 [SMILES: [2H]c1c(C(=O)O)c2cc(C)ccc2n1CC] | 2ae, 65% |
| 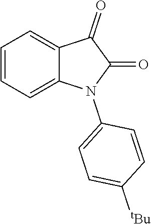 [SMILES: CC(C)(C)c1ccc(N2C(=O)C(=O)c3ccccc32)cc1] | 1af | 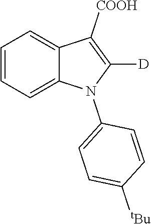 [SMILES: [2H]c1c(C(=O)O)c2ccccc2n1-c1ccc(C(C)(C)C)cc1] | 2af, 57% |

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

Following examples are given by way of illustration and therefore should not be construed to limit the scope of the present invention in any manner.

### Example 1

Preparation of Several N-Substituted Indole-3-Carboxylic Acid (Compound Represented by Formula 2b-e)

Into a 10 mL two neck round bottom flask equipped with an electromagnetic stirrer, transferred substituted indoline-2,3-dione (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired indole-3-carboxylic acid derivative (2b-e) in 55 to 65% yield.

### Example 2

Preparation of 1-Ethyl-Indole-3-Carboxylic Acid Derivative (Compound Represented by Formula 2f-k)

Into a 10 mL two neck round bottom flask equipped with an electromagnetic stirrer, transferred 1-ethyl indoline-2,3-dione derivative (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (26° C.) for 10 minutes. After that, the reaction mixture was warmed slowly to 80° C. and was kept for 2 hours. After the full conversion was monitored by TLC, it was cooled to room temperature (26° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl indole-3-carboxylic acid derivative (2f-k) in 53 to 83% yield.

### Example 3

### Preparation of 1-Methyl Indole-3-Carboxylic Acid (Compound Represented by Formula 21-p)

Into a 10 mL two neck round bottom flask equipped with an electromagnetic stirrer, transferred 1-methyl indoline-2,3-dione derivative (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-methyl indole-3-carboxylic acid derivative (21-p) in 38-64% yield.

### Example 4

Preparation of N-Aryl Indole-3-Carboxylic Acid (Compound Represented by Formula 2q-w)

Into a 10 mL two neck round bottom flask equipped with an electromagnetic stirrer, transferred 1-aryl indoline-2,3-dione (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (28° C.) for 10 minutes. After that, the reaction mixture was warmed slowly to 80° C. and was kept for 2 hours. After the full conversion was monitored by TLC, it was cooled to room temperature (28° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl-5-fluoro-1H-indole-3-carboxylic acid (2q-w) in 37 to 80% yield.

### Example 5

Preparation of 1-Aryl-1H-Indole-3-Carboxylic Acid Derivative (Compound Represented by Formula 2x-y)

Into a 10 mL two neck round bottom flask equipped with an electromagnetic stirrer, transferred 1-arylindoline-2,3-dione derivative (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-aryl-1H-indole-3-carboxylic acid derivative (2x-2y) in 66 to 80% yield.

### Example 6

### Preparation of 1H-Indole-3-Carboxylic Acid (Compound Represented by Formula 2z)

Into a 10 mL two neck round bottom flask equipped with an electromagnetic stirrer, transferred corresponding N-substituted indoline-2,3-dione (1 equiv., 0.2 mmol) and DMSO solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (28° C.) for 10 minutes. After that, the reaction mixture was warmed slowly to 80° C. and was kept for 2 hours. After the full conversion was monitored by TLC, it was cooled to room temperature (28° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1H-indole-3-carboxylic acid (2z) in 40-47% yield.

### Example 7

### Preparation of 1-Ethyl(¹³C—C2)1H-Indole-3-Carboxylic Acid (Compound Represented by Formula 2Aa)

Into a 10 mL two neck round bottom flask equipped with an electromagnetic stirrer, transferred 1-ethylindoline-2,3-dione (1 equiv., 0.2 mmol) and DMSO solvent (1 mL) and ¹³C DMSO solvent (0.1 mL) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (27° C.) for 10 minutes. After that, the reaction mixture was warmed slowly to 80° C. and was kept for 2 hours. After the full conversion was monitored by TLC, it was cooled to room temperature (27° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl(¹³C—C2)1H-indole-3-carboxylic acid (2aa) in 70% yield.

### Example 8

### Preparation of Several N-Substituted (C2-Deuterated) 1H-Indole-3-Carboxylic Acid Derivative (Compound Represented by Formula 2Ab-Ad)

Into a 10 mL two neck round bottom flask equipped with an electromagnetic stirrer, transferred substituted indoline-2,3-dione (1 equiv., 0.2 mmol) and DMSO-d₆ solvent (1 mL, 0.2 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired N-substituted (C₂-deuterated) 1H-indole-3-carboxylic acid derivative (2ab-ad) in 54 to 80% yield.

### Example 9

### Preparation of 1-Ethyl(C2-Deuterated)-Indole-3-Carboxylic Acid Derivative (Compound Represented by Formula 2Ae)

Into a 10 mL two neck round bottom flask equipped with an electromagnetic stirrer, transferred 1-ethyl-5-methylindoline-2,3-dione (1 equiv., 0.2 mmol) and DMSO-d₆ solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-ethyl(C2-deuterated) 1H-indole-3-carboxylic acid derivative (2ae) in 65% yield.

### Example 10

### Preparation of 1-(4-(Tert-Butyl)Phenyl) (C2-Deuterated)-Indole-3-Carboxylic Acid Derivative (Compound Represented by Formula 2Af)

Into a 10 mL two neck round bottom flask equipped with an electromagnetic stirrer, transferred 1-(4-(tert-butyl)phenyl)indoline-2,3-dione (1 equiv., 0.2 mmol) and DMSO-d₆ solvent (2 mL, 0.1 M) in an inert atmosphere. Then the whole reaction mixture was subjected to degassing by the Freeze-Pump-Thaw method. After degassing, NaH (6 equiv., 1.2 mmol) was added pinch-wise through an additional funnel and stirred at room temperature (30° C.) for 10 minutes. After that, the reaction mixture was warmed slowly to 80° C. and was kept for 2 hours. After the full conversion was monitored by TLC, it was cooled to room temperature (30° C.) and quenched with cold 1 N HCl solution (20 mL). The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired 1-(4-(tert-butyl)phenyl) (C₂-deuterated) 1H-indole-3-carboxylic acid derivative (2af) in 57% yield.

### Example 11

### Preparation of 5-H T3 Receptor Antagonist Tropisetron (4)

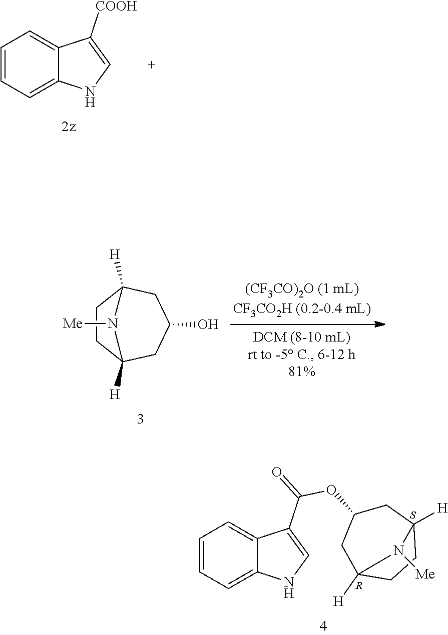

500 mg (3.1 mmol, 1 equiv.) indole-3-carboxylic acid (2z) was charged in an oven dried clean 25 mL two neck round bottom flask containing magnetic stir bar in N₂ atmosphere. 4 mL DCM and 0.2 mL (0.4 equiv)trifluoroacetic acid (TFA) was added respectively at room temperature (33° C.). After stirring for 5 minutes 1 mL (CF₃CO)₂O (7.2 mmol, 2.3 equiv.) was added dropwise in the reaction mixture at 0° C. Then it was warmed at rt and stirred for 2 h for activating the acid group. After that it was transferred to −5° C. and 4 mL tropine solution (3) (450 mg, 1 equiv.) (making previously by 4 mL DCM at inert condition) added dropwise for 30 minutes and was kept for 4 h. After full conversion checking by TLC, the whole reaction mixture was quenched by 100 mL ice-cooled 1 N NaOH solution followed by worked up with EtOAc, and brine. Then the organic layer was passed through Na₂SO₄, keeping for some time, and concentrated in a rotary evaporator. Then it was dissolved in EtOAc for crystallization, 710 mg (81%) desired tropisetron (4) obtained.

### Example 12

### Preparation of Imide N-Cyclohexyl-N-(Cyclohexylcarbamoyl)-1-Ethyl-1H-Indole-3-Carboxamide (5)

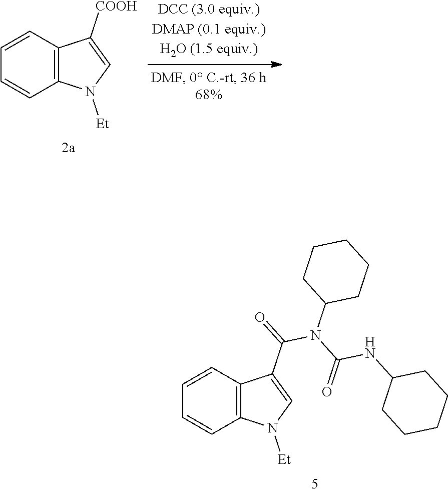 [SMILES: CCn1cc(C(=O)O)c2ccccc21 ; CCn1cc(C(=O)N(C(=O)NC2CCCCC2)C2CCCCC2)c2ccccc21]

Into a 10 mL two neck round bottom flask equipped with an electromagnetic stirrer, transferred N-ethyl indole-3-carboxylic acid (2a) (1 equiv., 0.3 mmol, 57 mg) and DMF solvent (4 mL, 0.075 M) in an inert atmosphere. The whole reaction mixture was subjected to cool down at 0° C. using crushed ice. After that DCC (3.0 equiv.), DMAP (0.1 equiv.), and H₂O (1.5 equiv.) was added slowly to the reaction mixture. Then it was warmed at room temperature (33° C.) and stirred for 36 h. After the full conversion was monitored by TLC, it was quenched with H₂O. The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (4:1) as eluent to obtain the desired imide compound 5 in 68% yield (81 mg).

### Example 13

### Preparation of Glucokinase Activator Amide 1-Ethyl-N-(Thiazol-2-Yl)-1H-Indole-3-Carboxamide (7)

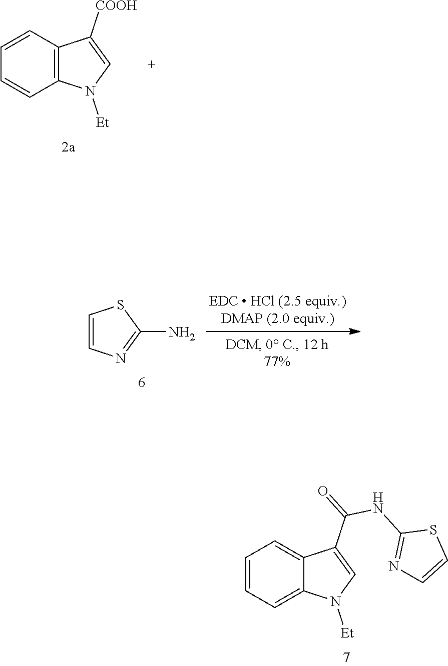 [SMILES: CCn1cc(C(=O)O)c2ccccc21 ; Nc1nccs1 ; CCn1cc(C(=O)Nc2nccs2)c2ccccc21]

Into a 10 mL two neck round bottom flask equipped with an electromagnetic stirrer, transferred N-ethyl indole-3-carboxylic acid (2a) (1 equiv., 0.4 mmol, 76 mg) and DCM solvent (4 mL, 0.1 M) followed by thiazol-2-amine (6) (1.1 equiv., 44 mg) in an inert atmosphere. The whole reaction mixture was subjected to cool down at 0° C. using crushed ice. After that EDC·HCl (2.5 equiv.) and DMAP (2 equiv.) was added slowly to the reaction mixture. Then it was warmed at room temperature (33° C.) and stirred for 12 h. After the full conversion was monitored by TLC, it was quenched with aqueous NaHCO₃. The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (2.3:1) as eluent to obtain the desired amide compound 7 in 77% yield (83 mg).

### Example 14

### Preparation of Directing Group Amide 1-Ethyl-N-(Quinolin-8-Yl)-1H-Indole-3-Carboxamide (9)

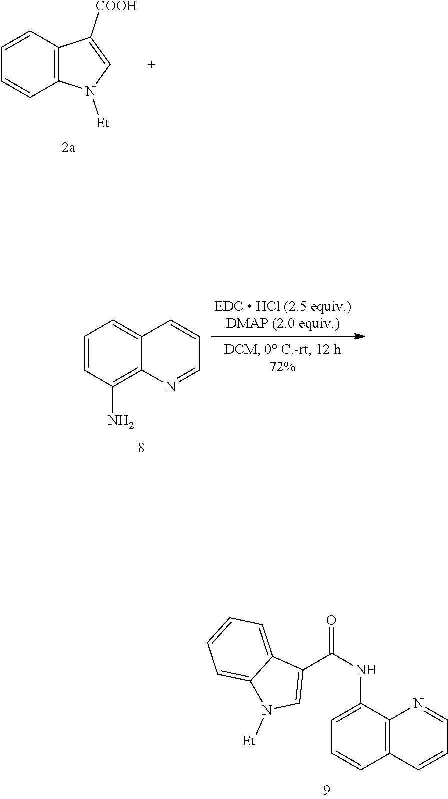 [SMILES: CCn1cc(C(=O)O)c2ccccc21 ; Nc1cccc2cccnc12 ; CCn1cc(C(=O)Nc2cccc3cccnc23)c2ccccc21]

Into a 10 mL two neck round bottom flask equipped with an electromagnetic stirrer, transferred N-ethyl indole-3-carboxylic acid (2a) (1 equiv., 0.4 mmol, 76 mg) and DCM solvent (4 mL, 0.1 M) followed by 8-aminoquinoline (8) (1.1 equiv., 64 mg) in an inert atmosphere. The whole reaction mixture was subjected to cool down at 0° C. using crushed ice. After that EDC·HCl (2.5 equiv.) and DMAP (2 equiv.) was added slowly to the reaction mixture. Then it was warmed at room temperature (33° C.) and stirred for 12 h. After the full conversion was monitored by TLC, it was quenched with aqueous NaHCO₃. The organic part was extracted by EtOAc (3×20 mL) with brine, dried over Na₂SO₄, and concentrated under reduced pressure. Finally, it was purified by column chromatography on silica gel (230-400 mesh) using petroleum ether/ethyl acetate (2.3:1) as eluent to obtain the desired amide compound 9 in 72% yield (91 mg).

### Advantages of the Invention

The main advantages of the present invention are as follows:

The present invention provides a simple, feasible, straightforward, economically viable, operationally simple one-pot process.

The synthesis method has a relatively simple operation, mild reaction conditions, high yield [up to 83%], and a simple process with moderate to good yield.

The method is less time consuming compared to existing methods.

Isatin has been used as a starting material which is cheaper than Indole/other designed starting materials.

No reducing agent, no transition metal, Lewis acid or costly reagents has been used.

The subsequent product separation process is very simple and it is useful for small-scale laboratory preparation as well as large-scale industrial production.

Subsequent product separation of this method uses work up (with EtOAc and brine) and crystallization methods, avoiding the existing methods where hazardous chemicals have been used in several steps giving moderate to low yield. Hence, the method is safer with a better yield compared to existing methods.

The process has been used to prepare commercially available drug such as Tropisetron in good yield and several other drugs such as Dolasetron etc can be made with low cost, high yield and suitable for industrial production.

Method is useful for large-scale synthesis purposes as bottle grade solvent (DMSO) performs very well where it is free from dissolved oxygen. At lower temperatures (60 to 70° C.) this conversion occurs yielding the desired product somewhat less than the optimum conditions.

The technical problem to be solved by the present invention is to overcome the existing synthetic method for preparing key starting material using high temperature and a large amount of solvent, complex multi-step operation, high cost of the chemicals and difficult to industrialize and other defects. Therefore, the method is cost-effective compared to existing methods.

The yields of the reported patented procedures were not mentioned clearly. In the present invention, various key starting materials have been produced in one step signifies that the invented methodology is viable for several functional groups. None of the expensive reagents, reactants, or solvents has been used for this novel transformation. It is wondering that bottle grade DMSO (should be free from dissolved oxygen) performs very well in this conversion. The present conversion happens at atmospheric pressure and it is operationally simple, economically viable, and effective for industrial preparation.

There is an urgent need for a better environmentally friendly and industrially viable methodology. The established method of the present invention provides a simple one-step strategy for synthesizing several ICA derivatives from easily available starting material Isatin employing NaH and DMSO or DMSO-d6 composite with high yield, simple reaction operation, and in mild reaction conditions. Its derivatives are widely used as artificial drugs. To improve the efficiency and practicability of this reaction, the present invention provides a simple and feasible new method with low cost, high yield, and suitable for industrial production.

Unique features of this reaction are the utilization of solvent (DMSO (Dimethylsulphoxide)/DMSO-d6) as a reactant and the formation ICA derivatives in good to excellent yields without using any reducing agents or transition metals.

I/We claim:

1. A process for the synthesis of indole-3-carboxylic acid (ICA) compound of Formula 2 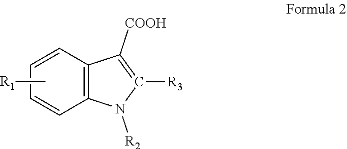
wherein
R₁ and R₂ are selected independently selected from the group consisting of hydrogen, linear or branched chain (C₁-C₁₂), perfluoro(C₁-C₁₂)alkyl, (C₃-C₁₂)cycloalkyl, (C₆-C₁₂)bicycloalkyl, (C₃-C₁₄)tricycloalkyl, (C₆-C₁₀)aryl, (C₆-C₁₀)aryl(C₁-C₆)alkyl, (C₁-C₆)alkyl(C₆-C₁₀)aryl, (C₆-C₁₀)aryl(C₁-C₃)alkoxy, perfluoro(C₆-C₁₀) aryl, perfluoro(C₆-C₁₀)aryl(C₁-C₃)alkyl, (C₅-C₁₀)heteroaryl, (C5-C10)heteroaryl(C₁-C₃)alkyl, hydroxy, (C₁-C₁₂)alkoxy, (C₃-C₁₂)cycloalkoxy, (C6-C12)bicycloalkoxy, (C₇-C₁₄)tricycloalkoxy, (C₆-C₁₀)aryloxy(C₁-C₃)alkyl, (C6-C10)aryloxy, (C₅-C₁₀)heteroaryloxy, (C₁-C₆)acyloxy, halogen, nitro and amino;
R₃ is selected from the group consisting of hydrogen, deuterium, and linear or branched chain (C₁-C₁₂);
C is carbon or ¹³ labelled carbon;
comprising the steps of:
i. degassing Isatin derivatives of Formula 1 and solvent of Formula 3 into a two neck round bottom flask equipped with an electromagnetic stirrer by the Freeze-Pump-Thaw method to obtain degassed mixture; 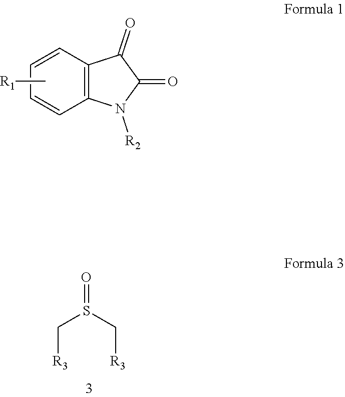
wherein R₁, R₂ and R₃ are same as above;
ii. adding sodium hydride [NaH] in the degassed mixture as obtained in step (i) at room temperature in the range of 25 to 35° C. for a period in the range of 5 to 10 minutes followed by warming the mixture slowly to 70 to 100° C. at one atmospheric pressure kept for period in the range of 1 to 24 hours to obtain a solution;
iii. cooling the solution as obtained in step (ii) at room temperature in the range of 25 to 35° C. followed by quenching with cold 1 N HCl solution;
iv. extracting the organic part by EtOAc with brine, dried over Na₂SO₄, and concentrated under reduced pressure, purified by column chromatography on silica gel using petroleum ether/ethyl acetate (4:1) as eluent to obtain the compound of Formula I.

2. The process as claimed in claim 1, wherein yield of the compound of Formula 2 is in the range of 50-83%.

3. A compound of Formula A 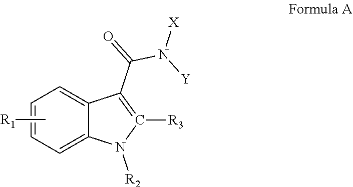
wherein
X═H or cyclohexyl;
Y═ 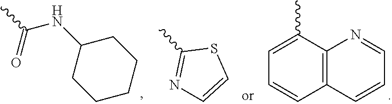
R₁ and R₂ are selected independently selected from the group consisting of hydrogen, linear or branched chain (C₁-C₁₂), perfluoro(C₁-C₁₂)alkyl, (C₃-C₁₂)cycloalkyl, (C₆-C₁₂)bicycloalkyl, (C₃-C₁₄)tricycloalkyl, (C₆-C₁₀)aryl, (C₆-C₁₀)aryl(C₁-C₆)alkyl, (C₁-C₆)alkyl(C₆-C₁₀)aryl, (C₆-C₁₀)aryl(C₁-C₃)alkoxy, perfluoro(C₆-C₁₀) aryl, perfluoro(C₆-C₁₀)aryl(C₁-C₃)alkyl, (C₅-C₁₀)heteroaryl, (C5-C10)heteroaryl(C₁-C₃)alkyl, hydroxy, (C₁-C₁₂)alkoxy, (C₃-C₁₂)cycloalkoxy, (C6-C12)bicycloalkoxy, (C₇-C₁₄)tricycloalkoxy, (C₆-C₁₀)aryloxy(C₁-C₃)alkyl, (C6-C10)aryloxy, (C₅-C₁₀)heteroaryloxy, (C₁-C₆)acyloxy, halogen, nitro and amino;
R₃ is selected from the group consisting of hydrogen, deuterium, and linear or branched chain (C₁-C₁₂);
C is carbon or ¹³ labeled carbon.

4. The compound as claimed in claim 3, wherein the compound of Formula A is selected from the group consisting of:
i. 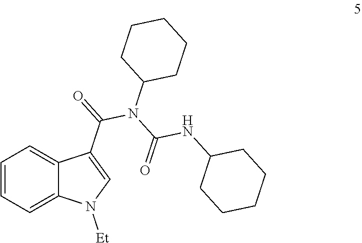 [SMILES: CCn1cc(C(=O)N(C(=O)NC2CCCCC2)C2CCCCC2)c2ccccc21]
ii. 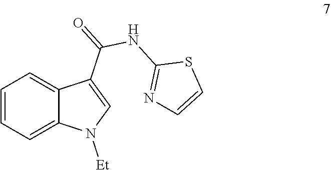 [SMILES: CCn1cc(C(=O)Nc2nccs2)c2ccccc21]
iii.

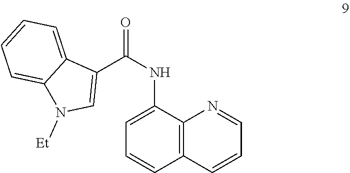 [SMILES: CCn1cc(C(=O)Nc2cccc3cccnc23)c2ccccc21]

5. The process as claimed in claim 1, wherein the compound of Formula 2 have been used to prepare commercially available drug Tropisetron (4) and potential bioactive indole compound of Formula A as claimed in claim 3 and 4.

6. The process as claimed in claim 1, wherein said process is used to prepare C13-labelled ICA compound of Formula 2aa.

7. The process as claimed in claim 1, wherein said process is used to prepare deuterated-ICA compound of Formula 2ab-2af.
